(19) United States
(12) Patent Application Publication
(10) Pub. No.: US 2024/0005696 A1
(43) Pub. Date: Jan. 4, 2024

(54) FINGER VEIN SENSORS AND METHODS OF USING THE SAME

(71) Applicant: NANJING EASTHOUSE ELECTRICAL CO., LTD., NANJING (CN)

(72) Inventor: HAO MIN, NANJING (CN)

(73) Assignee: NANJING EASTHOUSE ELECTRICAL CO., LTD., NANJING (CN)

(21) Appl. No.: 17/278,298

(22) PCT Filed: Mar. 19, 2021
(86) PCT No.: PCT/CN2021/081707

§ 371 Date: Mar. 20, 2021

(51) Int. Cl. G06V 40/145; G06V 10/141; H04N 23/23; H04N 23/67

(52) U.S. Cl. CPC G06V 40/145; G06V 10/141; H04N 23/23; H04N 23/675

(57) ABSTRACT

The present disclosure relates to several types of finger vein sensor. In certain embodiments, the finger vein sensor includes: an image sensor, and an infrared light source. Image sensor captures infrared image of finger vein pattern of a finger of a target human. The image sensor faces down and is positioned at top of finger vein sensor. The infrared light source may include a predetermined number of infrared light-emitting diodes (LED), and they are arranged in one or more rows and one or more columns and positioned at bottom of finger vein sensor. The finger is positioned between infrared light source and image sensor. The infrared light from the infrared light source irradiates the finger vertically from the bottom to generate the infrared image of finger vein pattern of the finger on the image sensor, and the image sensor captures the infrared image of finger vein pattern of the finger.

### FIELD

The present disclosure generally relates to user authentication, and more particularly to several finger vein sensors that provide better finger vein pattern and are contamination resistant, and the methods of using the finger vein sensors.

### BACKGROUND

Finger vein sensors have been widely used for user authentication. However, the conventional finger vein sensors have some issues that need to be improved. As shown in related art FIG. 10A and FIG. 10B, the finger vein sensor 1000 includes a sensor body 1001, a finger vein pattern sensing surface 1005, and an infrared light-emitting diode (LED) 1009. The infrared light-emitting diode (LED) 1009 irradiates infrared light on a finger 1007 and generates finger vein pattern on the finger vein pattern sensing surface 1005. The finger vein pattern sensing surface 1005 captures the finger vein pattern for user authentication. As shown in FIG. 10A and FIG. 10B, the finger vein pattern sensing surface 1005 is relatively small compared to the size of the finger 1007. Therefore, only a small portion of the finger vein pattern is captured for user authentication. The finger 1007 often touches the finger vein pattern sensing surface 1005, and any contamination on the surface of the finger 1007 may distort the finger vein pattern of the finger 1007 captured, which may cause authentication errors. Additionally, the sensor body 1001 defines a small space 1003 above the finger vein pattern sensing surface 1005. If the space 1003 has water on it, the finger vein sensor 1000 will fail. Therefore, the conventional finger vein sensors are widely used only in indoor applications.

Therefore, heretofore unaddressed needs still exist in the art to address the aforementioned deficiencies and inadequacies.

### SUMMARY

In one aspect, the present disclosure relates to a finger vein sensor. In certain embodiments, the finger vein sensor includes: an image sensor, an infrared light source, and a finger vein sensor controller. The image sensor is horizontally positioned at an upper end of the finger vein sensor. The image sensor faces down in vertical direction to capture at least one infrared image of finger vein pattern of a finger of a target human. The infrared light source is positioned at a lower end of the finger vein sensor. The infrared light source faces up and shines infrared light upward in vertical direction. The finger vein sensor controller includes a processor, and a non-volatile memory storing an operating system and computer executable instructions. The computer executable instructions include: an infrared light source control module, a finger vein verification module, an image/parameter storage module, an image processing module, an image sensor control module, and a finger vein sensor power module.

In certain embodiments, when executed at the processor, the computer executable instructions cause the processor to perform one or more of operations:
detecting, by the image sensor, that the finger is placed between the infrared light source and the image sensor;
irradiating, by the infrared light source from the lower end of the finger vein sensor, the infrared light through the finger to generate an infrared image of finger vein pattern of the finger on the image sensor;
detecting, by the image sensor, a set of infrared image quality parameters of the infrared image of finger vein pattern of the finger of the target human;
adjusting, by the infrared light source control module, a set of parameters of the infrared light source, until each of the set of infrared image quality parameters of the infrared image of finger vein pattern of the finger of the target human reaches a corresponding predetermined level; and
capturing, by the image sensor, the infrared image of finger vein pattern of the finger, and storing the captured infrared image of finger vein pattern of the finger into the image/parameter storage module.

In certain embodiments, the set of parameters of the infrared light source includes: infrared LED light intensity, infrared lighting directions, and infrared lighting orientations, and the set of infrared image quality parameters includes: clarity, brightness, contrast, noise distortion, and blurring distortion of the infrared image of finger vein pattern of the finger.

In certain embodiments, the finger vein sensor includes a finger vein sensor enclosure. The finger vein sensor includes a lower compartment and an upper compartment. In certain embodiments, the infrared light source is positioned in the lower compartment of the finger vein sensor enclosure, and the image sensor is positioned in the upper compartment of the finger vein sensor enclosure.

In certain embodiments, the infrared light source includes a group of infrared light-emitting diodes (LED), a group of infrared light bulbs, and/or any other infrared light sources. The group of infrared LED and the group of infrared light bulbs are arranged in one or more rows and one or more columns.

In certain embodiments, the infrared light source includes a lens. The lens is positioned between the finger and the image sensor in the upper compartment of the finger vein sensor enclosure. In certain embodiments, the lens includes an auto focus lens.

In certain embodiments, the infrared light source includes an infrared filter. The infrared filter is positioned between the lens and the image sensor in the upper compartment of the finger vein sensor enclosure for improving quality of the infrared image of finger vein pattern of the finger.

In certain embodiments, an upper surface of the lower compartment of the finger vein sensor enclosure forms a transparent finger resting surface for resting the finger on the transparent finger resting surface. A lower surface of the upper compartment of the finger vein sensor enclosure forms a transparent surface to allow the image sensor to capture the infrared image of finger vein pattern of the finger formed above the transparent finger resting surface.

In another aspect, the present disclosure relates to a finger vein sensor. In certain embodiments, the finger vein sensor includes an image sensor, an infrared light source, an optical reflector, and a finger vein sensor controller. The image sensor is positioned in an upper end of the finger vein sensor to capture at least one infrared image of finger vein pattern of a finger of a target human. The infrared light source is positioned at a lower end of the finger vein sensor. The infrared light source faces upward and shines infrared light upward in vertical direction. The optical reflector is positioned in an optical path between the image sensor and the infrared light source. The optical reflector reflects a vertically oriented infrared image of finger vein pattern of the finger to the horizontally oriented image sensor.

In certain embodiments, the finger vein sensor controller includes a processor, and a non-volatile memory. The non-volatile memory stores an operating system and computer executable instructions. The computer executable instructions include: an infrared light source control module, a finger vein verification module, an image/parameter storage module, an image processing module, an image sensor control module, and a finger vein sensor power module. When executed at the processor, the computer executable instructions cause the processor to perform one or more of operations:
detecting, by the image sensor, that the finger is placed between the infrared light source and the optical reflector;
irradiating, by the infrared light source from the lower end of the finger vein sensor, the infrared light through the finger to generate an infrared image of finger vein pattern of the finger on the image sensor through the optical reflector;
detecting, by the image sensor, a set of infrared image quality parameters of the infrared image of finger vein pattern of the finger of the target human;
adjusting, by the infrared light source control module, a set of parameters of the infrared light source, until each of the set of infrared image quality parameters of the infrared image of finger vein pattern of the finger of the target human reaches a corresponding predetermined level; and
capturing, by the image sensor, the infrared image of finger vein pattern of the finger, and storing the captured infrared image of finger vein pattern of the finger into the image/parameter storage module.

In certain embodiments, the set of parameters of the infrared light source includes: infrared LED light intensity, infrared lighting directions, and infrared lighting orientations, and the set of infrared image quality parameters includes: clarity, brightness, contrast, noise distortion, and blurring distortion of the infrared image of finger vein pattern of the finger.

In certain embodiments, the finger vein sensor includes a finger vein sensor enclosure. The finger vein sensor includes a lower compartment and an upper compartment. In certain embodiments, the infrared light source is positioned in the lower compartment of the finger vein sensor enclosure, and the image sensor is positioned in the upper compartment of the finger vein sensor enclosure.

In one embodiment, the image sensor is vertically positioned in a left side of the upper compartment of the finger vein sensor enclosure. The image sensor faces a center of the finger vein sensor in horizontal direction to capture the at least one infrared image of finger vein pattern of the finger of the target human through the optical reflector.

In another embodiment, the image sensor is vertically positioned in a right side of the upper compartment of the finger vein sensor enclosure. The image sensor faces a center of the finger vein sensor in horizontal direction to capture the at least one infrared image of finger vein pattern of the finger of the target human through the optical reflector.

In yet another embodiment, the image sensor is vertically positioned in a user-facing side of the upper compartment of the finger vein sensor enclosure. The image sensor faces a center of the finger vein sensor in horizontal direction to capture the at least one infrared image of finger vein pattern of the finger of the target human through the optical reflector.

In certain embodiments, the optical reflector includes a reflecting mirror, a triangular reflecting glass, or any other optical reflecting devices.

In certain embodiments, the infrared light source includes a group of infrared light-emitting diodes (LED), a group of infrared light bulbs, and/or any other infrared light sources. The group of infrared LED and the group of infrared light bulbs are arranged in one or more rows and one or more columns.

In certain embodiments, the infrared light source includes a lens. The lens is positioned between the finger and the image sensor in the upper compartment of the finger vein sensor enclosure. In certain embodiments, the lens includes an auto focus lens.

In certain embodiments, the infrared light source includes an infrared filter. The infrared filter is positioned between the lens and the image sensor in the upper compartment of the finger vein sensor enclosure for improving quality of the infrared image of finger vein pattern of the finger.

In certain embodiments, an upper surface of the lower compartment of the finger vein sensor enclosure forms a transparent finger resting surface for resting the finger on the transparent finger resting surface. A lower surface of the upper compartment of the finger vein sensor enclosure forms a transparent surface to allow the image sensor to capture the infrared image of finger vein pattern of the finger formed above the transparent finger resting surface.

In yet another aspect, the present disclosure relates to a method of using finger vein sensor. In certain embodiments, the method includes:
installing, by a user, a finger vein sensor. In certain embodiments, the finger vein sensor includes: an image sensor, an infrared light source, and a finger vein sensor controller. The finger vein sensor controller includes: a processor, and a non-volatile memory. The non-volatile memory stores an operating system and computer executable instructions. The computer executable instructions include: an infrared light source control module, a finger vein verification module, image/parameter storage module, an image processing module, an image sensor control module, and a finger vein sensor power module.
detecting, by the image sensor, that a finger of a target human is placed between the infrared light source and the image sensor;
irradiating, by the infrared light source from a lower compartment of a finger vein sensor enclosure, infrared light through the finger to generate an infrared image of finger vein pattern of the finger on the image sensor;
detecting, by the image sensor, a set of infrared image quality parameters of the infrared image of finger vein pattern of the finger of the target human;
adjusting, by the infrared light source control module, a set of parameters of the infrared light source, until each of the set of infrared image quality parameters of the infrared image of finger vein pattern of the finger of the target human reaches a corresponding predetermined level; and capturing, by the image sensor, the infrared image of finger vein pattern of the finger, and storing the captured infrared image of finger vein pattern of the finger into the image/parameter storage module.

In certain embodiments, the set of parameters of the infrared light source at least includes: infrared LED light intensity, infrared lighting directions, and infrared lighting orientations, and the set of infrared image quality parameters at least includes: clarity, brightness, contrast, noise distortion, and blurring distortion of the infrared image of finger vein pattern of the finger.

These and other aspects of the present disclosure will become apparent from the following description of the preferred embodiment taken in conjunction with the following drawings, although variations and modifications therein may be effected without departing from the spirit and scope of the novel concepts of the disclosure.

### BRIEF DESCRIPTION OF THE DRAWINGS

The accompanying drawings illustrate one or more embodiments of the present disclosure, and features and benefits thereof, and together with the written description, serve to explain the principles of the present invention. Wherever possible, the same reference numbers are used throughout the drawings to refer to the same or like elements of an embodiment, and wherein:

FIG. 1 illustrates a configuration of a finger vein sensor having an image sensor positioned on a top end of a finger vein sensor enclosure according to certain embodiments of the present disclosure;

FIG. 2 shows a block diagram of a finger vein sensor controller of a finger vein sensor 100 according to certain embodiments of the present disclosure;

FIG. 3 illustrates a configuration of a finger vein sensor having an image sensor positioned on a front facing side of the finger vein sensor enclosure and a reflecting mirror as an optical reflector according to certain embodiments of the present disclosure;

FIG. 4 illustrates a configuration of a finger vein sensor having an image sensor positioned on the front facing side of the finger vein sensor enclosure and a triangular reflecting glass as an optical reflector according to certain embodiments of the present disclosure;

FIG. 5 illustrates a configuration of a finger vein sensor having an image sensor positioned on a right side of the finger vein sensor enclosure and a reflecting mirror as an optical reflector according to certain embodiments of the present disclosure;

FIG. 6 illustrates a configuration of a finger vein sensor having an image sensor positioned on the right side of the finger vein sensor enclosure and the reflecting mirror as the optical reflector according to certain embodiments of the present disclosure;

FIG. 7 illustrates a configuration of a finger vein sensor having an image sensor positioned on a left side of the finger vein sensor enclosure and a triangular reflecting glass as an optical reflector according to certain embodiments of the present disclosure;

FIG. 8 shows a sectional view of a finger vein sensor having an image sensor positioned on the left side of the finger vein sensor enclosure and the triangular reflecting glass as the optical reflector according to certain embodiments of the present disclosure;

FIG. 9 shows a flowchart of a method of using the finger vein sensor to detect and capture one or more of the finger vein pattern images according to certain embodiments of the present disclosure; and

FIG. 10A shows a front view of a conventional finger vein sensor 1000, and FIG. shows a side sectional view of the conventional finger vein sensor 1000.

### DETAILED DESCRIPTION

The present disclosure is more particularly described in the following examples that are intended as illustrative only since numerous modifications and variations therein will be apparent to those skilled in the art. Various embodiments of the disclosure are now described in detail. Referring to the drawings, like numbers, if any, indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present disclosure. Additionally, some terms used in this specification are more specifically defined below.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the disclosure or of any exemplified term. Likewise, the disclosure is not limited to various embodiments given in this specification.

Unless otherwise defined, all technical and scientific terms used herein have the same meaning as commonly understood by one of ordinary skill in the art to which this disclosure pertains. In the case of conflict, the present document, including definitions will control.

As used herein, “around”, “about” or “approximately” shall generally mean within 20 percent, preferably within 10 percent, and more preferably within 5 percent of a
given value or range. Numerical quantities given herein are approximate, meaning that the term “around”, “about” or “approximately” can be inferred if not expressly stated.

As used herein, “plurality” means two or more.

As used herein, the terms “comprising,” “including,” “carrying,” “having,” “containing,” “involving,” and the like are to be understood to be open-ended, i.e., to mean including but not limited to.

As used herein, the phrase at least one of A, B, and C should be construed to mean a logical (A or B or C), using a non-exclusive logical OR. It should be understood that one or more steps within a method may be executed in different order (or conconventionally) without altering the principles of the present disclosure.

As used herein, the term module may refer to, be part of, or include an Application Specific Integrated Circuit (ASIC); an electronic circuit; a combinational logic circuit; a field programmable gate array (FPGA); an electronic key processor (shared, dedicated, or group) that executes code; other suitable hardware components that provide the described functionality; or a combination of some or all of the above, such as in a system-on-chip. The term module may include memory (shared, dedicated, or group) that stores code executed by the electronic key processor.

The term code, as used above, may include software, firmware, and/or microcode, and may refer to programs, routines, functions, classes, and/or objects. The term shared, as used above, means that some or all code from multiple modules may be executed using a single (shared) electronic key processor. In addition, some or all code from multiple modules may be stored by a single (shared) memory. The term group, as used above, means that some or all code from a single module may be executed using a group of electronic key processors. In addition, some or all code from a single module may be stored using a group of memories.

The apparatuses and methods described herein may be implemented by one or more computer programs executed by one or more electronic key processors. The computer programs include electronic key processor-executable instructions that are stored on a non-transitory tangible computer readable medium. The computer programs may also include stored data. Non-limiting examples of the non-transitory tangible computer readable medium are nonvolatile memory, magnetic storage, and optical storage.

The present disclosure will now be described more fully hereinafter with reference to the accompanying drawings, in which embodiments of the disclosure are shown. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. Like numbers refer to like elements throughout.

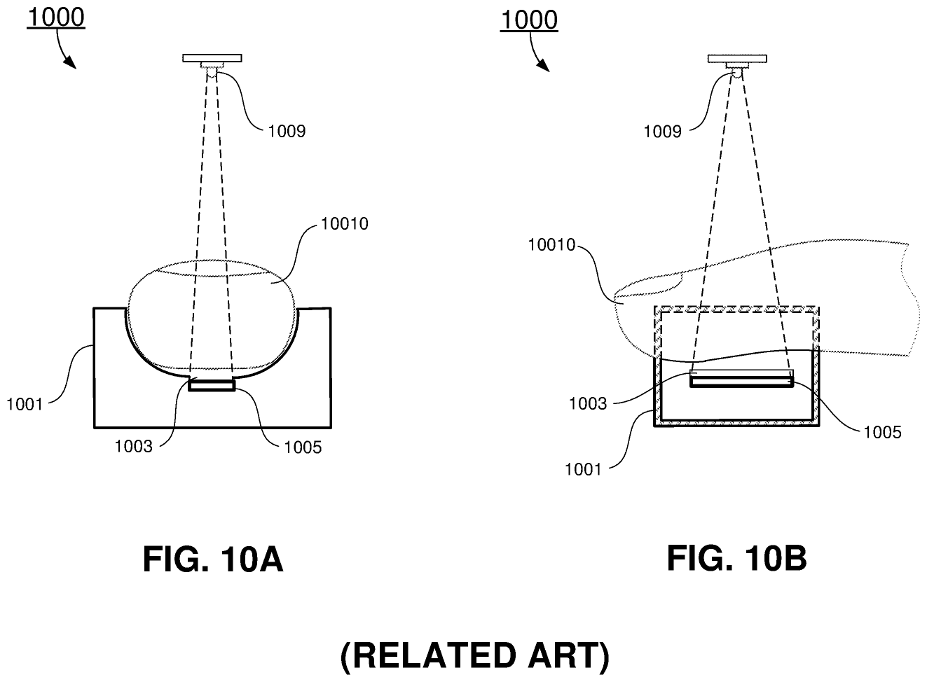

As shown in related art FIG. 10A and FIG. 10B, the conventional finger vein sensor 1000 has, among other things, following disadvantages. The finger vein pattern sensing surface 1005 is relatively small compared to the size of the finger 10010. Therefore, only a small portion of the finger vein pattern is captured for user authentication. The finger 10010 often touches the finger vein pattern sensing surface 1005, and any contamination on the surface of the finger 407 may distort the finger vein pattern of the finger 10010 captured, which may cause authentication errors. Additionally, the sensor body 1001 defines a small space 1003 above the finger vein pattern sensing surface 405. If the space 1003 has water in it, the finger vein sensor 1000 will fail. The present disclosure discloses several new improvements that will increase the size of the finger vein pattern images, that will eliminate authentication error caused by sensor surface contamination, and that will prevent authentication failures caused by moisture or water on the sensor surface.

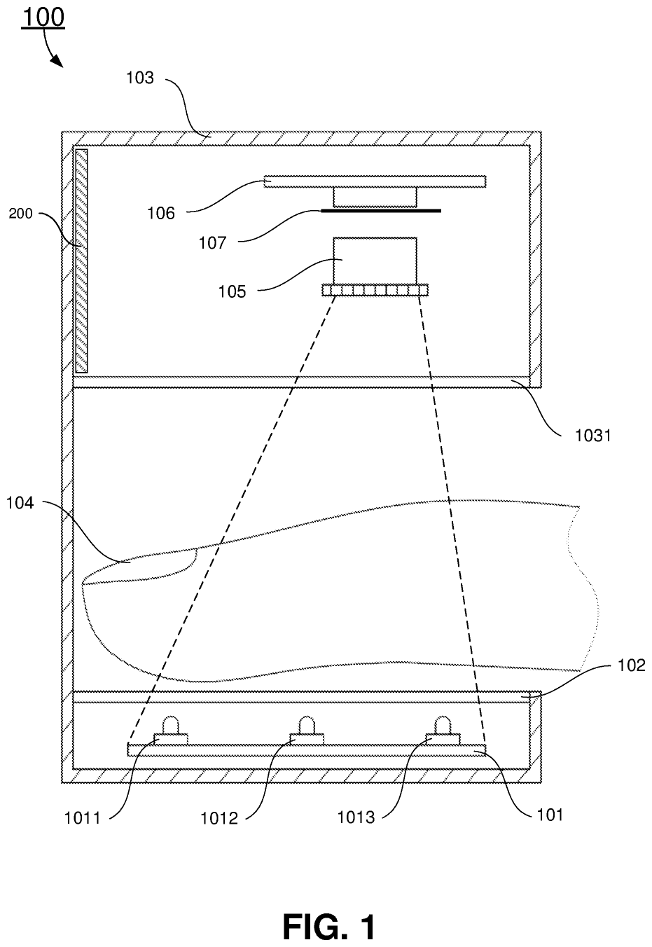

In one aspect, as shown in FIG. 1, the present disclosure relates to a finger vein sensor 100. In certain embodiments, the finger vein sensor 100 includes: an image sensor 106, an infrared light source 101, and a finger vein sensor controller 200. The image sensor 106 is horizontally positioned at an upper end of the finger vein sensor 100. The image sensor 106 faces down in vertical direction to capture at least one infrared image of finger vein pattern of a finger 104 of a target human. The infrared light source 101 is positioned at a lower end of the finger vein sensor 100. The infrared light source 101 faces up and shines infrared light upward in vertical direction.

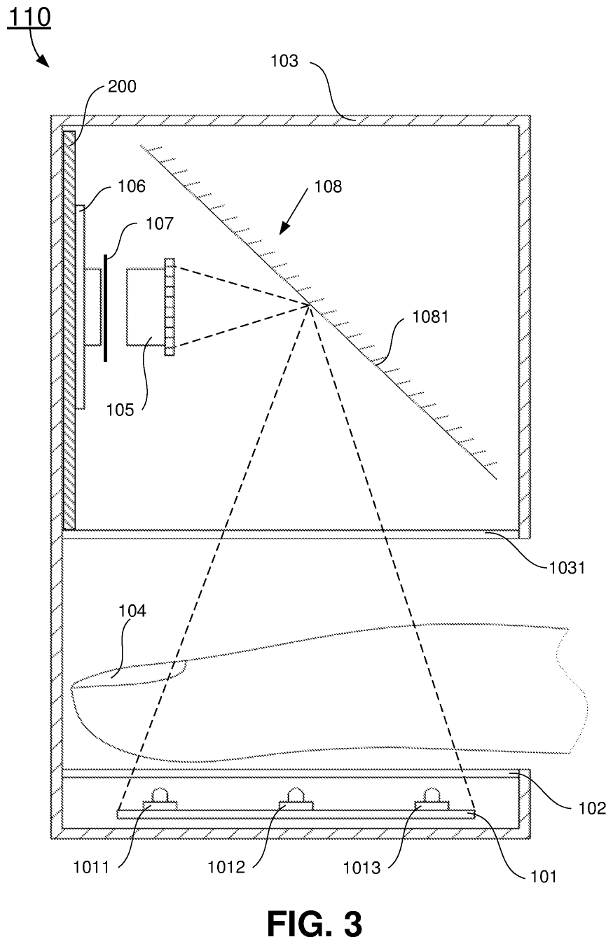

In certain embodiments, the infrared light source 101 includes a group of infrared light-emitting diodes (LED), a group of infrared light bulbs, and/or any other infrared light sources. In certain embodiments, in order to generate better quality of finger vein pattern images, the group of infrared LED and the group of infrared light bulbs are arranged in a matrix form having N rows 101N1, 101N2, . . . , and 101NM, and M columns 1011M, 1012M, . . . , and 101NM, as shown in FIG. 3-FIG. 8. In one embodiment, all N×M infrared LEDs can be lit up to generate finger vein pattern images. In another embodiment, some of N×M infrared LEDs can be lit up to generate finger vein pattern images. In an additional embodiment, each of the N rows of infrared LEDs can be lit up in turn to generate a scanning infrared light source 101 vertically. In yet another embodiment, each of the M column of infrared LEDs can be lit up in turn to generate another scanning infrared light source 101 horizontally. These variations of lighting patterns are created to alter parameters of infrared light source 101 such as LED light intensity, lighting directions, and lighting orientations to generate better quality finger vein pattern images.

In certain embodiments, the finger vein sensor controller 200 includes a processor 202, and a non-volatile memory 204. The non-volatile memory 204 stores an operating system 2042 and computer executable instructions 2044. The computer executable instructions 2044 include: an infrared light source control module 20441, a finger vein verification module 20442, an image/parameter storage module 20443, an image processing module 20444, an image sensor control module 20445, and a finger vein sensor power module 20446. The infrared light source control module 20441 controls the infrared light source 101, creates various infrared lighting patterns to alter the parameters of infrared light source 101 such as LED light intensity, lighting directions, and lighting orientations to generate better quality finger vein pattern images. The finger vein verification module 20442 verifies identity of the target human using the finger vein pattern images captured by the image sensor 106. The image/parameter storage module 20443 stores the finger vein pattern images captured by the image sensor 106, as well as various parameters of the infrared light source 101. The image processing module 20444 processes the captured finger vein pattern images, and based on the processing results, the lighting patterns of the infrared light source 101 is changed through the infrared light source control module 20441 to generate improved image quality of the finger vein pattern images. The image sensor control module 20445 controls exposure sensitivity, exposure timing and exposure sequence of the image sensor 106 to capture improved image quality of the finger vein pattern images.

In certain embodiments, the finger vein sensor power module 20446 provides electrical power to the finger vein sensor 100 for supporting the operation of the finger vein sensor 100. In one embodiment, the finger vein sensor power module 20446 may receive electrical power from an external source, such as an alternate current (AC) electrical source, or a direct current (DC) electrical source. In another embodiment, the finger vein sensor power module 20446 may receive electrical power from a battery. In yet another embodiment, the finger vein sensor power module 20446 may receive electrical power from a rechargeable battery. The rechargeable battery includes at least one of: a lead-acid rechargeable battery, a nickel cadmium (NiCd) rechargeable battery, a nickel metal hydride (NiMH) rechargeable battery, a lithium ion (Li-ion) rechargeable battery, and a lithium ion polymer (Li-ion polymer) rechargeable battery.

In certain embodiments, when executed at the processor 202, the computer executable instructions 2044 cause the processor 202 to perform one or more of operations:
detecting, by the image sensor 106, that the finger 104 is placed between the infrared light source 101 and the image sensor 106;
irradiating, by the infrared light source 101 from the lower end of the finger vein sensor 100, the infrared light through the finger 104 to generate an infrared image of finger vein pattern of the finger 104 on the image sensor 106;
detecting, by the image sensor 106, a set of infrared image quality parameters of the infrared image of finger vein pattern of the finger 104 of the target human;
adjusting, by the infrared light source control module 20441, a set of parameters of the infrared light source 101, until each of the set of infrared image quality parameters of the infrared image of finger vein pattern of the finger 104 of the target human reaches a corresponding predetermined level; and
capturing, by the image sensor 106, the infrared image of finger vein pattern of the finger 104, and storing the captured infrared image of finger vein pattern of the finger 104 into the image/parameter storage module 20443.

In certain embodiments, the set of parameters of the infrared light source 101 includes: infrared LED light intensity, infrared lighting directions, and infrared lighting orientations, and the set of infrared image quality parameters includes: clarity, brightness, contrast, noise distortion, and blurring distortion of the infrared image of finger vein pattern of the finger 104.

In certain embodiments, as shown in FIG. 1, the finger vein sensor 100 includes a finger vein sensor enclosure 103. The finger vein sensor enclosure 103 includes a lower compartment and an upper compartment. The infrared light source 101 is positioned in the lower compartment and provides infrared light through a transparent finger resting surface 102 vertically from the lower compartment.

In certain embodiments, as shown in FIG. 1, the finger vein sensor 100 includes a lens 105. The lens 105 is positioned between the finger 104 and the image sensor 106 in the upper compartment of the finger vein sensor enclosure 103. In certain embodiments, the lens 105 includes an auto focus lens.

In certain embodiments, as shown in FIG. 1, the finger vein sensor 100 includes an infrared filter 107. The infrared filter 107 is positioned between the lens 105 and the image sensor 106 in the upper compartment of the finger vein sensor enclosure 103 for improving quality of the infrared image of finger vein pattern of the finger 104.

In certain embodiments, as shown in FIG. 1, an upper surface of the lower compartment of the finger vein sensor enclosure 103 forms a transparent finger resting surface 102 for resting the finger 104 on the transparent finger resting surface 102. This transparent finger resting surface 102 allows the target human to rest the finger 104 on it and generates a steady finger vein pattern image at a fixed location. It prevents inconsistency when the finger 104 moves up and down.

In certain embodiments, as shown in FIG. 1, a lower surface of the upper compartment of the finger vein sensor enclosure 103 forms a transparent surface 1031 to allow the image sensor 106 to capture the infrared image of finger vein pattern of the finger 104 formed above the transparent finger resting surface 102.

The lens 105 is positioned between the finger 104 and the image sensor 106. The lens 105 is adjusted to focus on the finger vein pattern generated above the transparent finger resting surface 102. The combination of the lens 105 and the transparent finger resting surface 102 allows the image sensor 106 to capture consistent finger vein pattern images and to improve quality of the infrared image of finger vein pattern of the finger 104.

In certain embodiments, the infrared filter 107 is positioned between the lens 105 and the image sensor 106 for improving quality of the infrared image of finger vein pattern of the finger 104. The infrared filter 107 allows infrared lights to pass and eliminates light interference from any lights other than infrared light. Therefore, the application of the infrared filter 107 also improves the quality of the infrared image of finger vein pattern of the finger 104.

The configuration of the finger vein sensor 100 creates larger size finger vein pattern images than the conventional finger vein sensors. Conventional finger vein sensor allows user to touch the image forming surface of the finger vein sensor, any contamination on the image forming surface of the finger vein sensor will be captured by the conventional vein sensor and it will cause authentication errors. The finger vein sensor 100 prevents such errors from happening. Any contaminations such as dirt on the surface of the finger 104, the dirt accumulated on the transparent finger resting surface 102, or finger prints left on the transparent finger resting surface 102 will not be captured by the image sensor 106. The water stains or water accumulated on the transparent finger resting surface 102 will not cause any authentication errors because the water will be transparent and will not distort the finger vein pattern of the finger 104.

As shown in FIG. 1, in order to create large finger vein pattern image, the image sensor 106 should have sufficient distance from the finger 104 resting on the transparent finger resting surface 102 because of the straight infrared light path from the bottom to the top of the finger vein sensor enclosure 103. This may cause the finger vein sensor enclosure 103 to become tall. In order to shorten the height of the finger vein sensor enclosure 103, a few more exemplary embodiments of finger vein sensors are described as following. In certain embodiments, the straight infrared light path from the bottom to the top of the finger vein sensor enclosure 103 may be reflected by an optical reflector to become a horizontal infrared light path.

In another aspect, as shown in FIGS. 3-8, the present disclosure relates to a finger vein sensor 100. In certain embodiments, the finger vein sensor 100 includes an image sensor 106, an infrared light source 101, an optical reflector 108, and a finger vein sensor controller 200. The image sensor 106 is positioned in an upper end of the finger vein sensor 100 to capture at least one infrared image of finger vein pattern of a finger 104 of a target human. The infrared light source 101 is positioned at a lower end of the finger vein sensor 100. The infrared light source 101 faces upward and shines infrared light upward in vertical direction. The optical reflector 108 is positioned in an optical path between the image sensor 106 and the infrared light source 101. The optical reflector 108 reflects a vertically oriented infrared image of finger vein pattern of the finger 104 to the horizontally oriented image sensor 106.

In certain embodiments, as shown in FIGS. 3-8, the infrared light source 101 includes a group of infrared light-emitting diodes (LED), a group of infrared light bulbs, and/or any other infrared light sources. In certain embodiments, in order to generate better quality of finger vein pattern images, the group of infrared LED and the group of infrared light bulbs are arranged in a matrix form having N rows 101N1, 101N2, . . . , and 101NM, and M columns 1011M, 1012M, . . . , and 101NM, as shown in FIG. 3-FIG. 8. In one embodiment, all N×M infrared LEDs can be lit up to generate finger vein pattern images. In another embodiment, some of N×M infrared LEDs can be lit up to generate finger vein pattern images. In an additional embodiment, each of the N rows of infrared LEDs can be lit up in turn to generate a scanning infrared light source 101 vertically. In yet another embodiment, each of the M column of infrared LEDs can be lit up in turn to generate another scanning infrared light source 101 horizontally. These variations of lighting patterns are created to alter parameters of infrared light source 101 such as LED light intensity, lighting directions, and lighting orientations to generate better quality finger vein pattern images.

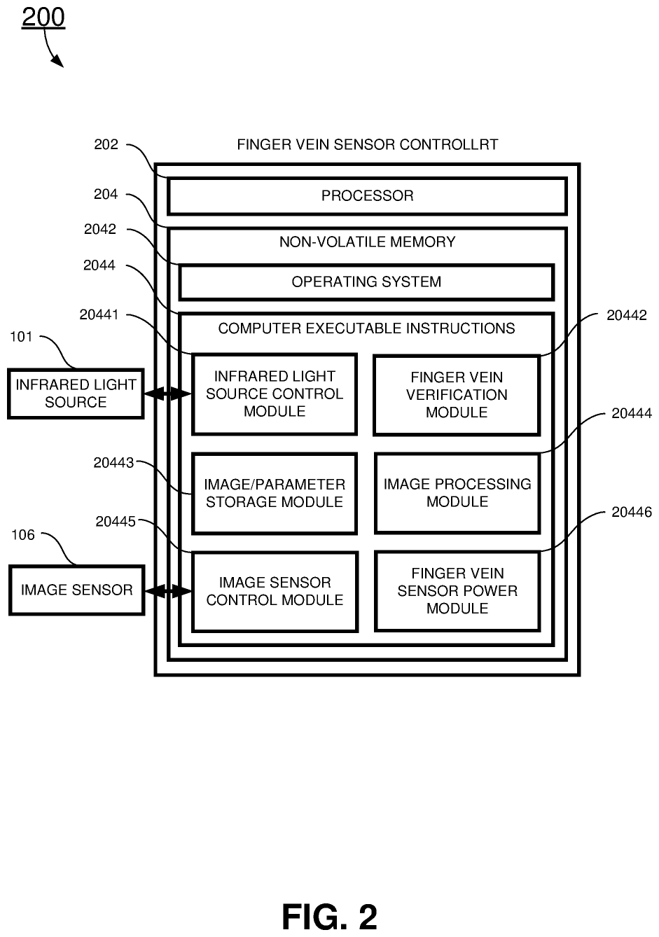

In certain embodiments, as shown in FIG. 2, the finger vein sensor controller 200 includes a processor 202, and a non-volatile memory 204. The non-volatile memory 204 stores an operating system 2042 and computer executable instructions 2044. The computer executable instructions 2044 include: an infrared light source control module 20441, a finger vein verification module 20442, an image/parameter storage module 20443, an image processing module 20444, an image sensor control module 20445, and a finger vein sensor power module 20446. The infrared light source control module 20441 controls the infrared light source 101, creates various infrared lighting patterns to alter the parameters of infrared light source 101 such as LED light intensity, lighting directions, and lighting orientations to generate better quality finger vein pattern images. The finger vein verification module 20442 verifies identity of the target human using the finger vein pattern images captured by the image sensor 106. The image/parameter storage module 20443 stores the finger vein pattern images captured by the image sensor 106, as well as various parameters of the infrared light source 101. The image processing module 20444 processes the captured finger vein pattern images, and based on the processing results, the lighting patterns of the infrared light source 101 is changed through the infrared light source control module 20441 to generate improved image quality of the finger vein pattern images. The image sensor control module 20445 controls exposure sensitivity, exposure timing and exposure sequence of the image sensor 106 to capture improved image quality of the finger vein pattern images.

In certain embodiments, as shown in FIG. 2, the finger vein sensor power module 20446 provides electrical power to the finger vein sensor 100 for supporting the operation of the finger vein sensor 100. In one embodiment, the finger vein sensor power module 20446 may receive electrical power from an external source, such as an alternate current (AC) electrical source, or a direct current (DC) electrical source. In another embodiment, the finger vein sensor power module 20446 may receive electrical power from a battery. In yet another embodiment, the finger vein sensor power module 20446 may receive electrical power from a rechargeable battery. The rechargeable battery includes at least one of: a lead-acid rechargeable battery, a nickel cadmium (NiCd) rechargeable battery, a nickel metal hydride (NiMH) rechargeable battery, a lithium ion (Li-ion) rechargeable battery, and a lithium ion polymer (Li-ion polymer) rechargeable battery.

In certain embodiments, when executed at the processor 202, the computer executable instructions 2044 cause the processor 202 to perform one or more of operations:
detecting, by the image sensor 106, that the finger 104 is placed between the infrared light source 101 and the optical reflector 108;
irradiating, by the infrared light source 101 from the lower end of the finger vein sensor 100, the infrared light through the finger 104 to generate an infrared image of finger vein pattern of the finger 104 on the image sensor 106 through the optical reflector 108;
detecting, by the image sensor 106, a set of infrared image quality parameters of the infrared image of finger vein pattern of the finger 104 of the target human;
adjusting, by the infrared light source control module 20441, a set of parameters of the infrared light source 101, until each of the set of infrared image quality parameters of the infrared image of finger vein pattern of the finger 104 of the target human reaches a corresponding predetermined level; and
capturing, by the image sensor 106, the infrared image of finger vein pattern of the finger 104, and storing the captured infrared image of finger vein pattern of the finger 104 into the image/parameter storage module 20443.

In certain embodiments, the set of parameters of the infrared light source 101 includes: infrared LED light intensity, infrared lighting directions, and infrared lighting orientations, and the set of infrared image quality parameters includes: clarity, brightness, contrast, noise distortion, and blurring distortion of the infrared image of finger vein pattern of the finger 104.

In certain embodiments, as shown in FIGS. 3-8, the finger vein sensor 100 includes a finger vein sensor enclosure 103. The finger vein sensor 100 includes a lower compartment and an upper compartment. In certain embodiments, the infrared light source 101 is positioned in the lower compartment of the finger vein sensor enclosure 103, and the image sensor 106 is positioned in the upper compartment of the finger vein sensor enclosure 103.

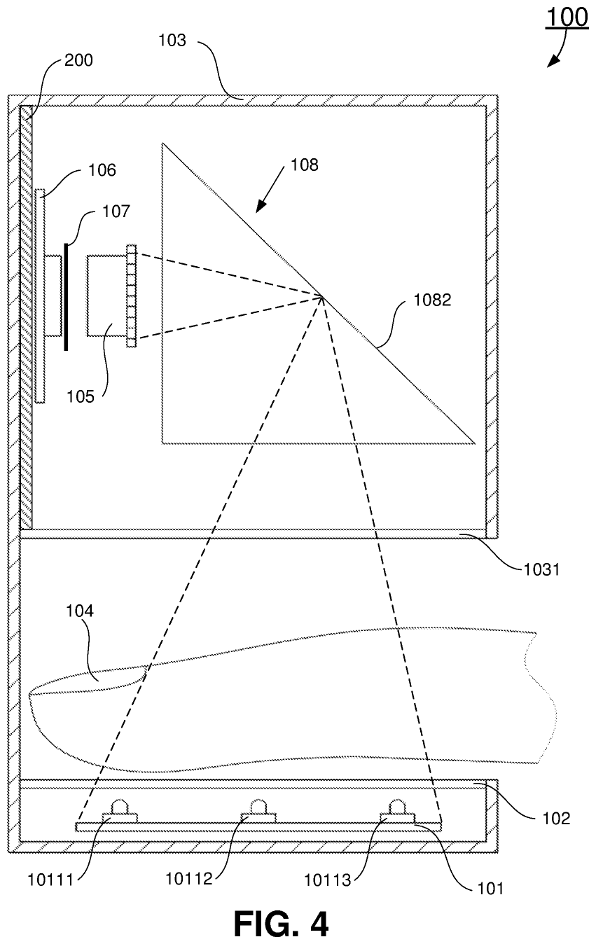

In one embodiment, as shown in FIGS. 3-4, the image sensor 106 is vertically positioned in a user-facing side of the upper compartment of the finger vein sensor enclosure 103. The image sensor 106 faces a center of the finger vein sensor 100 in horizontal direction to capture the at least one infrared image of finger vein pattern of the finger 104 of the target human through the optical reflector 108.

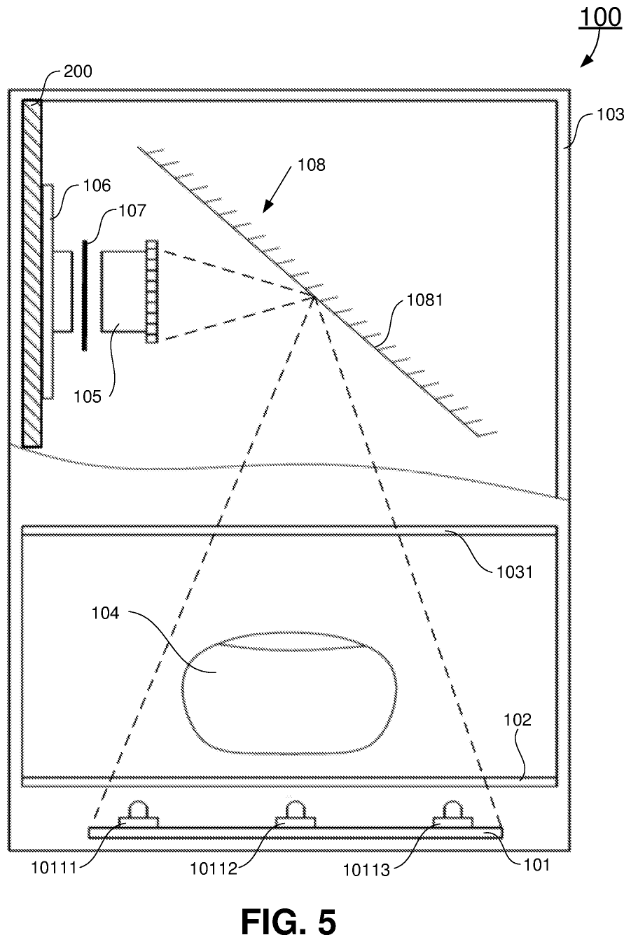

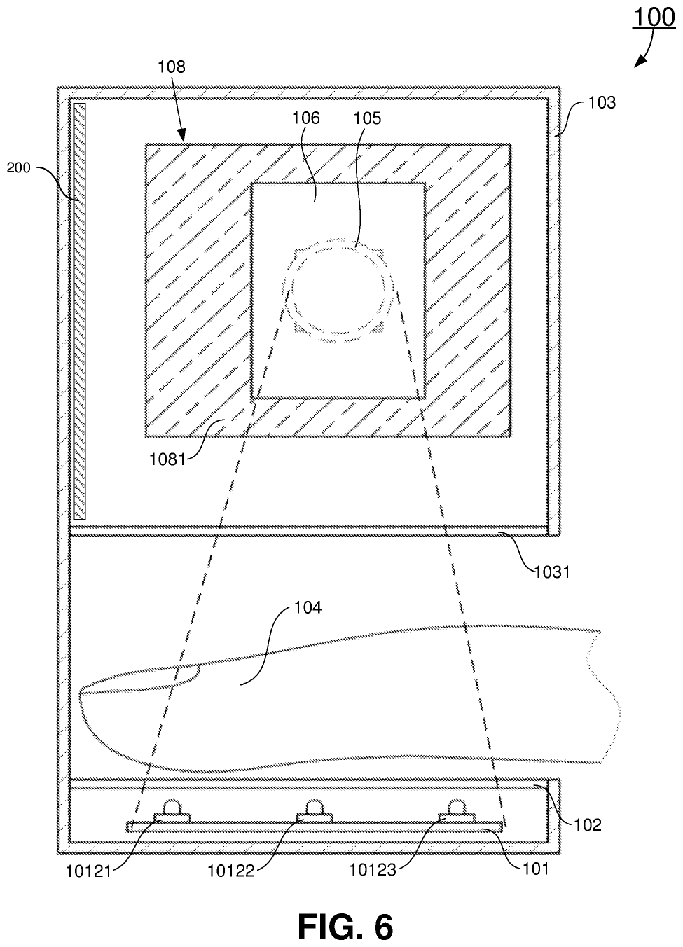

In another embodiment, as shown in FIGS. 5-6, the image sensor 106 is vertically positioned in a right side of the upper compartment of the finger vein sensor enclosure 103. The image sensor 106 faces a center of the finger vein sensor 100 in horizontal direction to capture the at least one infrared image of finger vein pattern of the finger 104 of the target human through the optical reflector 108.

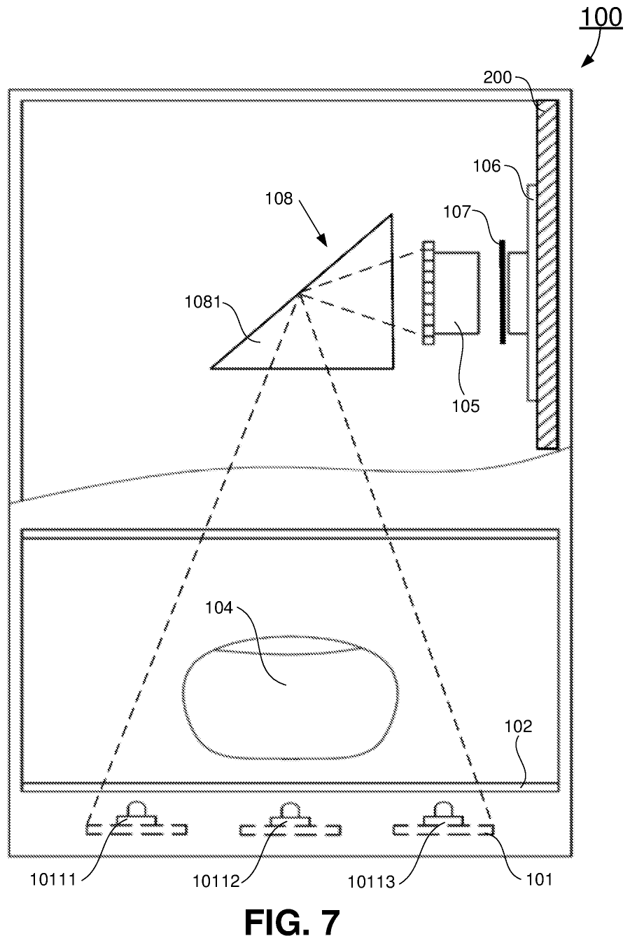

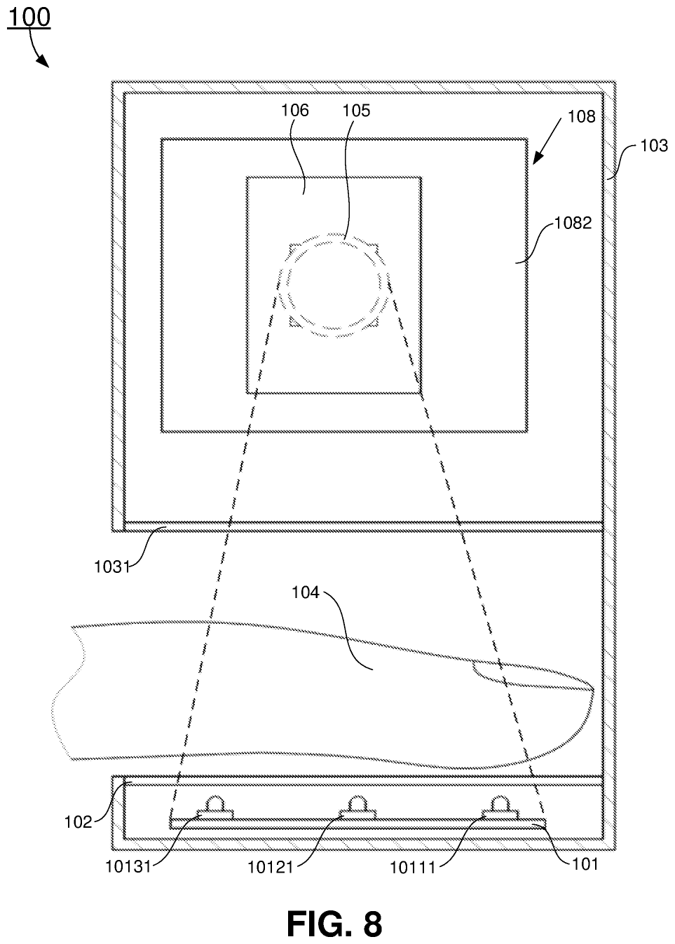

In one embodiment, as shown in FIGS. 7-8, the image sensor 106 is vertically positioned in a left side of the upper compartment of the finger vein sensor enclosure 103. The image sensor 106 faces a center of the finger vein sensor 100 in horizontal direction to capture the at least one infrared image of finger vein pattern of the finger 104 of the target human through the optical reflector 108.

In certain embodiments, the optical reflector 108 includes a reflecting mirror 1081, as shown in FIGS. 3 and 5, a triangular reflecting glass 1082, as shown in FIGS. 4 and 8, or any other optical reflecting devices.

In certain embodiments, as shown in FIGS. 3-8, the finger vein sensor 100 includes a lens 105. The lens 105 is positioned between the finger 104 and the image sensor 106 in the upper compartment of the finger vein sensor enclosure 103. In certain embodiments, the lens 105 includes an auto focus lens.

In certain embodiments, as shown in FIGS. 3, 4, 5 and 7, the finger vein sensor 100 includes an infrared filter 107. The infrared filter 107 is positioned between the lens 105 and the image sensor 106 in the upper compartment of the finger vein sensor enclosure 103 for improving quality of the infrared image of finger vein pattern of the finger 104.

In certain embodiments, as shown in FIGS. 3-8, an upper surface of the lower compartment of the finger vein sensor enclosure 103 forms a transparent finger resting surface 102 for resting the finger 104 on the transparent finger resting surface 102. This transparent finger resting surface 102 allows the target human to rest the finger 104 on it and generates a steady finger vein pattern image at a fixed location. It prevents inconsistency when the finger 104 moves up and down.

In certain embodiments, as shown in FIGS. 3-8, a lower surface of the upper compartment of the finger vein sensor enclosure 103 forms a transparent surface 1031 to allow the image sensor 106 to capture the infrared image of finger vein pattern of the finger 104 formed above the transparent finger resting surface 102.

The lens 105 is positioned between the finger 104 and the image sensor 106. The lens 105 is adjusted to focus on the finger vein pattern generated above the transparent finger resting surface 102. The combination of the lens 105 and the transparent finger resting surface 102 allows the image sensor 106 to capture consistent finger vein pattern images and to improve quality of the infrared image of finger vein pattern of the finger 104.

In certain embodiments, the infrared filter 107 is positioned between the lens 105 and the image sensor 106 for improving quality of the infrared image of finger vein pattern of the finger 104. The infrared filter 107 allows infrared lights to pass and eliminates light interference from any lights other than infrared light. Therefore, the application of the infrared filter 107 also improves the quality of the infrared image of finger vein pattern of the finger 104.

The configuration of the finger vein sensor 100 creates larger size finger vein pattern images than the conventional finger vein sensors. Conventional finger vein sensor allows user to touch the image forming surface of the finger vein sensor, any contamination on the image forming surface of the finger vein sensor will be captured by the conventional vein sensor and it will cause authentication errors. The finger vein sensor 100 prevents such errors from happening. Any contaminations such as dirt on the surface of the finger 104, the dirt accumulated on the transparent finger resting surface 102, or finger prints left on the transparent finger resting surface 102 will not be captured by the image sensor 106. The water stains or water accumulated on the transparent finger resting surface 102 will not cause any authentication errors because the water will be transparent and will not distort the finger vein pattern of the finger 104.

In yet another aspect, the present disclosure relates to a method of using finger vein sensor 100. In certain embodiments, the method includes:
installing, by a user, a finger vein sensor 100. In certain embodiments, the finger vein sensor 100 includes: an image sensor 106, an infrared light source 101, and a finger vein sensor controller 200. The finger vein sensor controller 200 includes: a processor 202, and a non-volatile memory 204. The non-volatile memory 204 stores an operating system 2042 and computer executable instructions 2044. The computer executable instructions 2044 include: an infrared light source control module 20441, a finger vein verification module 20442, image/parameter storage module 20443, an image processing module 20444, an image sensor control module 20445, and a finger vein sensor power module 20446.
detecting, by the image sensor 106, that a finger 104 of a target human is placed between the infrared light source 101 and the image sensor 106;
irradiating, by the infrared light source 101 from a lower compartment of a finger vein sensor enclosure 103, infrared light through the finger 104 to generate an infrared image of finger vein pattern of the finger 104 on the image sensor 106;
detecting, by the image sensor 106, a set of infrared image quality parameters of the infrared image of finger vein pattern of the finger 104 of the target human;
adjusting, by the infrared light source control module 20441, a set of parameters of the infrared light source 101, until each of the set of infrared image quality parameters of the infrared image of finger vein pattern of the finger 104 of the target human reaches a corresponding predetermined level; and
capturing, by the image sensor 106, the infrared image of finger vein pattern of the finger 104, and storing the captured infrared image of finger vein pattern of the finger 104 into the image/parameter storage module 20443.

In certain embodiments, the set of parameters of the infrared light source 101 at least includes: infrared LED light intensity, infrared lighting directions, and infrared lighting orientations, and the set of infrared image quality parameters at least includes: clarity, brightness, contrast, noise distortion, and blurring distortion of the infrared image of finger vein pattern of the finger 104.

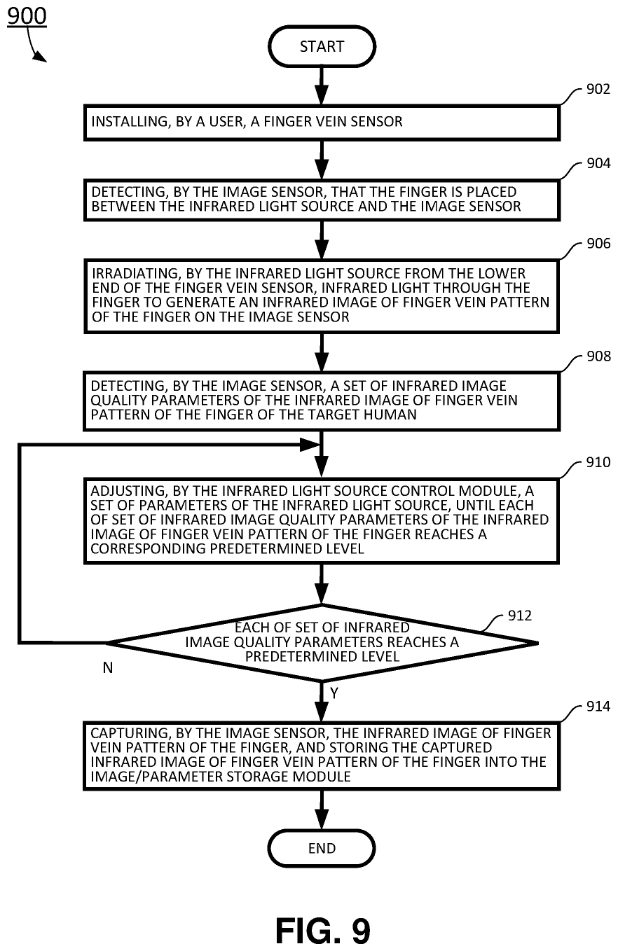

Referring now to FIG. 9, a flowchart of the method 900 of using a finger vein sensor 100 to detect and capture one or more of the finger vein pattern images is shown according to certain embodiments of the present disclosure.

At block 902, installing, by a user, a finger vein sensor 100. In certain embodiments, the finger vein sensor 100 includes: an image sensor 106, an infrared light source 101, and a finger vein sensor controller 200. The finger vein sensor controller 200 includes: a processor 202, and a non-volatile memory 204. The non-volatile memory 204 stores an operating system 2042 and computer executable instructions 2044. The computer executable instructions 2044 include: an infrared light source control module 20441, a finger vein verification module 20442, image/parameter storage module 20443, an image processing module 20444, an image sensor control module 20445, and a finger vein sensor power module 20446.

At block 904, detecting, by the image sensor 106, a finger 104 of a target human is placed between the infrared light source 101 and the image sensor 106.

At block 906, irradiating, by the infrared light source 101 from a lower compartment of a finger vein sensor enclosure 103, infrared light through the finger 104 to generate an infrared image of finger vein pattern of the finger 104 on the image sensor 106.

At block 908, detecting, by the image sensor 106, a set of infrared image quality parameters of the infrared image of finger vein pattern of the finger 104 of the target human. In certain embodiments, the set of infrared image quality parameters includes: clarity, brightness, contrast, noise distortion, and blurring distortion of the infrared image of finger vein pattern of the finger 104.

At block 910, adjusting, by the infrared light source control module 20441, a set of parameters of the infrared light source 101, until each of the set of infrared image quality parameters of the infrared image of finger vein pattern of the finger 104 of the target human reaches a corresponding predetermined level. In certain embodiments, the set of parameters of the infrared light source 101 includes: infrared LED light intensity, infrared lighting directions, and infrared lighting orientations.

At query block 912, checking, by the image processing module 20444, whether each of the set of infrared image quality parameters of the infrared image of finger vein pattern of the finger 104 of the target human reaches a corresponding predetermined level. When each of the set of infrared image quality parameters of the infrared image of finger vein pattern of the finger 104 of the target human reaches a corresponding predetermined level, the method proceeds to block 914. Otherwise, the method proceeds to block 910 and continues adjusting the set of the parameters of the infrared light source 101.

At block 914, capturing, by the image sensor 106, the infrared image of finger vein pattern of the finger 104, and storing the captured infrared image of finger vein pattern of the finger 104 into the image/parameter storage module 20443.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope. Accordingly, the scope of the present disclosure is defined by the appended claims rather than the foregoing description and the exemplary embodiments described therein.

What is claimed is:

1. A finger vein sensor comprising:
an image sensor horizontally positioned at an upper end of the finger vein sensor, wherein the image sensor faces down in vertical direction to capture at least one infrared image of finger vein pattern of a finger of a target human;
an infrared light source positioned at a lower end of the finger vein sensor, wherein the infrared light source faces up and shines infrared light upward in vertical direction; and
a finger vein sensor controller, wherein the finger vein sensor controller comprises a processor, and a non-volatile memory storing an operating system and computer executable instructions, wherein the computer executable instructions comprise: an infrared light source control module, a finger vein verification module, image/parameter storage module, an image processing module, an image sensor control module, and a finger vein sensor power module, wherein when executed at the processor, the computer executable instructions cause the processor to perform one or more of operations:
detecting, by the image sensor, that the finger is placed between the infrared light source and the image sensor;
irradiating, by the infrared light source from the lower end of the finger vein sensor, the infrared light through the finger to generate an infrared image of finger vein pattern of the finger on the image sensor;
detecting, by the image sensor, a plurality of infrared image quality parameters of the infrared image of finger vein pattern of the finger of the target human;
adjusting, by the infrared light source control module, a plurality of parameters of the infrared light source, until each of the plurality of infrared image quality parameters of the infrared image of finger vein pattern of the finger of the target human reaches a corresponding predetermined level; and
capturing, by the image sensor, the infrared image of finger vein pattern of the finger, and storing the captured infrared image of finger vein pattern of the finger into the image/parameter storage module.

2. The finger vein sensor of claim 1, wherein the plurality of parameters of the infrared light source at least comprises: infrared LED light intensity, infrared lighting directions, and infrared lighting orientations, and the plurality of infrared image quality parameters at least comprises: clarity, the brightness, the contrast, the noise distortion, and the blurring distortion of the infrared image of finger vein pattern of the finger.

3. The finger vein sensor of claim 1, further comprising a finger vein sensor enclosure having a lower compartment for positioning the infrared light source and an upper compartment for positioning the image sensor.

4. The finger vein sensor of claim 3, wherein the infrared light source comprises a plurality of infrared light-emitting diodes (LED), a plurality of infrared light bulbs, and/or any other infrared light sources, wherein the plurality of infrared LED and the plurality of infrared light bulbs are arranged in one or more rows and one or more columns.

5. The finger vein sensor of claim 3, further comprising a lens positioned between the finger and the image sensor in the upper compartment of the finger vein sensor enclosure, wherein the lens comprises an auto focus lens.

6. The finger vein sensor of claim 3, further comprising an infrared filter positioned between the lens and the image sensor in the upper compartment of the finger vein sensor enclosure for improving quality of the infrared image of finger vein pattern of the finger.

7. The finger vein sensor of claim 3, wherein the lower compartment of the finger vein sensor enclosure comprises a transparent finger resting surface for resting the finger on the transparent finger resting surface, and a lower surface of the upper compartment of the finger vein sensor enclosure forms a transparent surface to allow the image sensor to capture the infrared image of finger vein pattern of the finger formed above the transparent finger resting surface.

8. A finger vein sensor comprising:
an image sensor, wherein the image sensor is positioned in an upper end of the finger vein sensor to capture at least one infrared image of finger vein pattern of a finger of a target human;
an infrared light source positioned at a lower end of the finger vein sensor; wherein the infrared light source faces upward and shines infrared light upward in vertical direction;
an optical reflector positioned in an optical path between the image sensor and the infrared light source, wherein the optical reflector reflects a vertically oriented infrared image of finger vein pattern of the finger to the horizontally oriented image sensor; and
a finger vein sensor controller, wherein the finger vein sensor controller comprises a processor, and a non-volatile memory storing an operating system and computer executable instructions, wherein the computer executable instructions comprise: an infrared light source control module, a finger vein verification module, image/parameter storage module, an image processing module, an image sensor control module, and a finger vein sensor power module, wherein when executed at the processor, the computer executable instructions cause the processor to perform one or more of operations:
detecting, by the image sensor, that the finger is placed between the infrared light source and the optical reflector;
irradiating, by the infrared light source from the lower end of the finger vein sensor, the infrared light through the finger to generate an infrared image of finger vein pattern of the finger on the image sensor through the optical reflector;
detecting, by the image sensor, a plurality of infrared image quality parameters of the infrared image of finger vein pattern of the finger of the target human;
adjusting, by the infrared light source control module, a plurality of parameters of the infrared light source, until each of the plurality of infrared image quality parameters of the infrared image of finger vein pattern of the finger of the target human reaches a corresponding predetermined level; and
capturing, by the image sensor, the infrared image of finger vein pattern of the finger, and storing the captured infrared image of finger vein pattern of the finger into the image/parameter storage module.

9. The finger vein sensor of claim 8, wherein the plurality of parameters of the infrared light source at least comprises: infrared LED light intensity, infrared lighting directions, and infrared lighting orientations, and the plurality of infrared image quality parameters at least comprises: clarity, the brightness, the contrast, the noise distortion, and the blurring distortion of the infrared image of finger vein pattern of the finger.

10. The finger vein sensor of claim 8, further comprising a finger vein sensor enclosure having a lower compartment for positioning the infrared light source and an upper compartment for positioning the image sensor.

11. The finger vein sensor of claim 10, wherein the image sensor is vertically positioned in a left side of the upper compartment of the finger vein sensor enclosure, wherein the image sensor faces a center of the finger vein sensor in horizontal direction to capture the at least one infrared image of finger vein pattern of the finger of the target human through the optical reflector.

12. The finger vein sensor of claim 10, wherein the image sensor is vertically positioned in a right side of the upper compartment of the finger vein sensor enclosure, wherein the image sensor faces the center of the finger vein sensor in horizontal direction to capture the at least one infrared image of finger vein pattern of the finger of the target human through the optical reflector.

13. The finger vein sensor of claim 10, wherein the image sensor is vertically positioned in a user-facing side of the upper compartment of the finger vein sensor enclosure, wherein the image sensor faces the center of the finger vein sensor in horizontal direction to capture at least one infrared image of finger vein pattern of the finger of the target human through the optical reflector;

14. The finger vein sensor of claim 10, wherein the infrared light source comprises a plurality of infrared light-emitting diodes (LED), a plurality of infrared light bulbs, and/or any other infrared light sources, wherein the plurality of infrared LED and the plurality of infrared light bulbs are arranged in one or more rows and one or more columns.

15. The finger vein sensor of claim 10, further comprising a lens positioned between the optical reflector and the image sensor in the upper compartment of the finger vein sensor enclosure, wherein the lens comprises an auto focus lens.

16. The finger vein sensor of claim 10, further comprising an infrared filter positioned between the lens and the image sensor in the upper compartment of the finger vein sensor enclosure for improving quality of the infrared image of finger vein pattern of the finger.

17. The finger vein sensor of claim 10, wherein the optical reflector comprises a reflecting mirror, a triangular reflecting glass, or any other optical reflecting devices.

18. The finger vein sensor of claim 10, wherein the lower compartment of the finger vein sensor enclosure comprises a transparent finger resting surface for resting the finger on the transparent finger resting surface, and a lower surface of the upper compartment of the finger vein sensor enclosure forms a transparent surface to allow the image sensor to capture the infrared image of finger vein pattern of the finger formed above the transparent finger resting surface.

19. A method of using a finger vein sensor, comprising:
installing, by a user, the finger vein sensor, wherein the finger vein sensor comprises: an image sensor, an infrared light source, and a finger vein sensor controller, and the finger vein sensor controller comprises: a processor, and a non-volatile memory storing an operating system and computer executable instructions, wherein the computer executable instructions comprise: an infrared light source control module, a finger vein verification module, image/parameter storage module, an image processing module, an image sensor control module, and a finger vein sensor power module;
detecting, by the image sensor, that a finger of a target human is placed between the infrared light source and the image sensor;
irradiating, by the infrared light source from a lower compartment of a finger vein sensor enclosure, infrared light through the finger to generate an infrared image of finger vein pattern of the finger on the image sensor;
detecting, by the image sensor, a plurality of infrared image quality parameters of the infrared image of finger vein pattern of the finger of the target human;
adjusting, by the infrared light source control module, a plurality of parameters of the infrared light source, until each of the plurality of infrared image quality parameters of the infrared image of finger vein pattern of the finger of the target human reaches a corresponding predetermined level; and
capturing, by the image sensor, the infrared image of finger vein pattern of the finger, and storing the captured infrared image of finger vein pattern of the finger into the image/parameter storage module.

20. The method of claim 19, wherein the plurality of parameters of the infrared light source at least comprises: infrared LED light intensity, infrared lighting directions, and infrared lighting orientations, and the plurality of infrared image quality parameters at least comprises: clarity, the brightness, the contrast, the noise distortion, and the blurring distortion of the infrared image of finger vein pattern of the finger.
